(19) United States
(12) Patent Application Publication
(10) Pub. No.: US 2003/0215871 A1
(43) Pub. Date: Nov. 20, 2003

(54) Chemically inert molecular tags

(75) Inventors: John J. Baldwin, Gwynedd Valley, PA (US); Roland E. Dolle III, King of Prussia, PA (US); Lawrence W. Dillard, Skillman, NJ (US); Joan Guo, Plainsboro, NJ (US)

Correspondence Address: HESLIN ROTHENBERG FARLEY & MESITI PC, 5 COLUMBIA CIRCLE, ALBANY, NY, 12203, US

(73) Assignee: PHARMACOPEIA, INC., Cranbury, NJ (US)

(21) Appl. No.: 10/137,139

(22) Filed: May 1, 2002

(51) Int. Cl. G01N 33/53; C07C 69/52; G01N 33/543

(52) U.S. Cl. 435/7.1; 436/518; 560/223

(57) ABSTRACT

A method for identifying a solid support in combinatorial synthesis or determining the structure of a compound in combinatorial synthesis. The method involves attaching, detaching and identifying at least one tag by detaching the tag from the solid support and reacting the detached tag with a fluoroalkanoyl acid or its alcohol-reactive equivalent. A preferred linker/tag is of formula

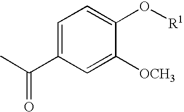

in which R¹ is C₁₃ to C₃₃ alkane or ether.

### TECHNICAL FIELD

This invention relates generally to the synthesis of chemical compounds, and more particularly, to the solid phase synthesis of combinatorial libraries of chemical compounds.

### BACKGROUND OF THE INVENTION

Combinatorial organic synthesis is becoming an important tool in drug discovery. Methods for the synthesis of large numbers of diverse compounds have been described [Ellman, et. al. Chem. Rev. 96: 555-600 (1996)], as have methods for tagging systems [Ohlmeyer et al., Proc. Natl. Acad. Sci. USA, 90, 10922-10926, (1993)]. The growing importance of combinatorial synthesis has created a need for new tags having chemical properties to accommodate a wide range of synthetic conditions and physical properties to allow detection at very low levels of sample.

Identifier tags, in their most general form, are means whereby one can identify which synthon has been incorporated onto an individual solid support in the synthesis of a compound. The identifier tag also records the step in the synthesis series in which the solid support visited that reaction. Identifier tags are defined in U.S. Pat. No. 5,708,153, col 4, lines 24-36, as “any recognizable feature which is, for example: microscopically distinguishable in shape, size, color, optical density, etc.; differently absorbing or emitting of light; chemically reactive; magnetically or electronically encoded; or in some other way distinctively marked with the required information, and decipherable at the level of one (or few) solid support(s).”

Molecular tags are a subset of identifier tags. Molecular tags are chemical entities which possess several properties: they are detachable from the solid supports, preferably by means orthogonal to those employed for releasing the compound of pharmacological interest; they are stable under the synthetic conditions; and they are capable of being detected at very low concentrations, e.g., 10⁻¹⁸ to 10⁻⁹ mole. Suitable molecular tags and methods for their employment are described in U.S. Pat. No. 5,565,324, the entire disclosure of which is incorporated herein by reference.

Known, commonly employed molecular tags, such as amines (described in U.S. Pat. No. 5,846,324), peptides, nucleotides (described in U.S. Pat. No. 5,708,153) and polychlorophenoxyalkyl tags (described in U.S. Pat. No. 5,565,324) suffer substantial degradation in the presence of certain reagents that one might wish to employ in combinatorial synthesis. Strong nucleophiles, such as thiols and alkoxide anions, and organometallic reagents, such as Grignard reagents and alkyllithium reagents, are problematic, even for the more robust tags. It would be useful to have a tag that allows detection in less than nanomolar amounts and that would withstand more aggressive reaction conditions in combinatorial synthesis than do the tags of the art.

### SUMMARY OF THE INVENTION

The present invention relates to a method of tagging that demonstrates the ability to withstand many common reaction conditions that would be desirable in combinatorial synthesis and is detectable at sub-nanomolar levels.

In one aspect, the invention relates to a method for identifying a solid support in combinatorial synthesis or determining the reaction history of a compound in combinatorial synthesis. From the reaction history, one can determine the structure of the compound. The method comprises attaching, detaching and identifying at least one tag by detaching the tag from the solid support and reacting the detached tag with a detection reagent. Since the tag is an alkane or oxaalkane attached by an oxygen to the solid support, detaching it generates an alkanol or oxaalkanol. The tag will usually be attached to the solid support through an intervening linker, such that the oxygen linkage occurs between the tag and the linker, and the linker is attached to the solid support. Detection reagents are chosen from fluoroalkanoyl acids and their alcohol-reactive equivalents. Alcohol-reactive equivalents, as the term is used herein, are synthons that can deliver an acyl residue to an alcohol. They include anhydrides, halides, and activated esters, as described below.

In one embodiment, the invention relates to a method for identifying a solid support in combinatorial synthesis comprising:

(a) attaching at least one alkane or oxaalkane tag via an oxygen linkage to the solid support;

(b) carrying out at least one chemical reaction on the solid support with the tag attached;

(c) detaching the tag from the solid support;

(d) reacting the detached tag with a detection reagent chosen from fluoroalkanoyl acids, anhydrides, halides, and activated esters, to provide an alkyl fluoroalkanoate or oxaalkyl fluoroalkanoate ester detectable tag; and

(e) detecting the tag, whereby the solid support is identified.

In another embodiment, the invention relates to a method for determining the structure of a compound, which comprises:

(a) providing a solid support on which a compound was synthesized by a reaction series comprising at least a first stage reagent and/or first stage reaction condition, and a second stage reagent and/or second stage reaction condition, wherein the first stage precedes the second stage in the reaction series, which solid support has attached thereto:

i. a first alkane or oxaalkane tag, which tag comprises a code that records the first stage reagent or the first stage reaction condition; and

ii. a second alkane or oxaalkane tag, which tag comprises a code that records the second stage reagent or the second stage reaction condition;

(b) detaching the tags from the solid support such that a mixture of alkanol or oxaalkanol tags is formed;

(c) reacting the mixture of tags with a detection reagent chosen from fluoroalkanoyl acids, anhydrides, halides, and activated esters to form a mixture of alkyl fluoroalkanoate or oxaalkyl fluoroalkanoate esters; and

(d) detecting each tag.

In another aspect, the invention relates to a linker/tag combination that is particularly useful in the method of the invention:

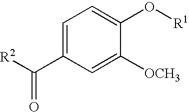

wherein

R¹ is C₁₃ to C₃₃ alkane or ether; and

R² is chosen from —CHN₂, —OH, halogen, —O-succinimide, and —O-pentafluorophenyl.

In the following disclosure, the variables are defined when introduced and retain that definition throughout.

### DETAILED DESCRIPTION OF THE INVENTION

“Alkyl” is intended to include linear, cyclic or branched hydrocarbon structures and combinations thereof of 1 to 30 carbons. “Lower alkyl” means alkyl groups of from 1 to 6 carbon atoms. Examples of lower alkyl groups include methyl, ethyl, propyl, isopropyl, butyl, s-and t-butyl, pentyl, hexyl, and the like. “Cycloalkyl” is a subset of alkyl that refers to saturated hydrocarbons of from 3 to 12 carbon atoms having one or more rings. Examples of “cycloalkyl” groups include c-propyl, c-butyl, c-pentyl,c-hexyl, 2-methylcyclopropyl, cyclopropylmethyl, cyclopentylmethyl, norbornyl, adamantyl, myrtanyl and the like.

The C₁₃ to C₃₃ ether residue, also known as oxaalkyl, refers to alkyl as described above, in which one or more carbons (with its associated hydrogens) is replaced by oxygen. Examples would include 14-ethoxytetradecanyl, 3,6,9,12,15-tetraoxaheptadecanyl and the like.

“Halo” includes F, Cl and Br.

“Fluoroalkyl” refers to an alkyl residue in which one or more hydrogen atoms are replaced with F, for example: trifluoromethyl, 4,4,4-trifluorobutyl, and pentafluoroethyl.

For the purpose of the present invention, the term combinatorial library means a collection of molecules based on logical design and involving the selective combination of building blocks by means of simultaneous chemical reactions. Each species of molecule in the library is referred to as a member of the library.

Linkers are commonly used in combinatorial synthesis to attach tags as well as to attach the moiety of putative chemical or pharmacological interest. Linkers are molecules that can be attached to a solid support and to which either the tags of the invention or the desired members of a library of chemical compounds may be attached. When the construction of the library is complete, the linker allows clean separation of the target compounds and the tags from the solid support without harm to the compounds and preferably without damage to the support. Many linkers have been described in the literature. Suitable linkers are disclosed in U.S. Pat. No. 5,789,172, the disclosure of which is incorporated herein by reference.

The materials upon which the combinatorial syntheses are performed are referred to variously as solid phase supports, solid supports, beads, and resins. These terms are intended to include:

(a) beads, pellets, disks, fibers, gels, or particles such as cellulose beads, pore-glass beads, silica gels, polystyrene beads optionally cross-linked with divinylbenzene and optionally grafted with polyethylene glycol, poly-acrylamide beads, latex beads, dimethylacrylamide beads optionally cross-linked with N,N′-bis-acryloyl ethylene diamine, glass particles coated with hydrophobic polymer, etc., i.e., material having a rigid or semi-rigid surface; and (b) soluble supports such as polyethylene glycol or low molecular weight, non-cross-linked polystyrene. The solid supports may, and usually do, have surfaces that have been functionalized with amino, hydroxy, carboxy, or halo groups; amino groups are most common. Techniques for functionalizing the surface of solid phases are well known in the art. Attachment of lysine to the surface of an amine-functionalized bead (to increase the number of available sites) and subsequent attachment of linkers as well as further steps in a typical combinatorial synthesis are described, for example, in PCT application WO95/30642, the disclosure of which is incorporated herein by reference. In the synthesis described in WO95/30642, the linker is a photolytically cleavable linker.

As discussed above, in its broad aspect, the invention relates to a method for identifying a solid support in combinatorial synthesis or determining the structure of a compound comprising attaching, detaching and identifying at least one alkane or oxaalkane tag. Usually one will employ more than one alkane or oxaalkane tag, two or more such tags being more common. Identification is accomplished by reacting an alkanol or oxaalkanol, obtained by detaching the tag from the solid support, with a detection reagent chosen from fluoroalkanoyl acids and their alcohol-reactive equivalents.

An important feature of the invention is that it relates to a tag that need not be detectable at 10 nanomoles or less (an “undetectable tag”) before reacting the detached tag with a detection reagent, but which becomes detectable after derivatization. “Undetectable” means that the presence or absence of the tag, either attached to the solid support or in the reaction mixture following cleavage, cannot be established with statistical significance (without further chemical modification or derivatization) by the detector in question. “Undetectable”, in its global sense thus means that the presence or absence of the tag cannot be established with statistical significance by any detector available at the time of filing this patent application. This feature allows one to employ as tags highly chemically inert residues, such as alkanes and ethers, because the chemically more sensitive functionality that will allow detection is added after the construction of the combinatorial library is complete.

In the electrophoric tags described in U.S. Pat. No. 5,789,172, the polychlorinated aromatic ring of the tag is the salient electrophoric element required for electron capture gas chromatography (ECGC) detection and analysis. Unfortunately, it is this structural feature that is responsible for the tag's chemical instability in the presence of certain desirable reagents. Decreasing the number of chlorine atoms on the ring can attenuate chemical instability; the CₓCl₃ tags are somewhat less reactive that the CₓCl₅ tags. In principal, a series of CₓCl₂ tags or CₓCl tags would be more stable than CₓCl₃ tags, but reducing the number of Cl atoms renders the tags undetectable by EC.

Non-halogenated tags, such as those based on aliphatic alcohols, are ideal in terms of their chemical stability. Unlike the aryl C—Cl bond, the aliphatic C—H bond is unreactive toward reducing reagents, radicals, Pd-catalyzed carbon-carbon and carbon-nitrogen bond couplings, metal-halogen exchange, and strong nucleophiles. However, aliphatic alcohols are non-electrophoric. According to the invention, aliphatic alcohols and ethers are made electron-capturing by derivatization with an appropriate reagent. In the process of the invention, aliphatic-based tags are removed (by oxidation in the examples) and converted to their polyfluoroalkanoyl esters or ethers prior to ECGC analysis. Polyfluoroalkanoyl esters and ethers are readily detected by ECGC and are cheap and simple to synthesize. Thus, fully robust, chemically resistant tags are utilized during combinatorial synthesis, and derivatized to their respective electrophoric ethers or esters after cleavage.

Non-halogenated alcohols are well suited as inert tags. Selection criteria for optimal tags are multi-fold. The tag alcohols should be either commercially available or readily synthesized. The purity of each tag must be sufficient to afford a clean signal in the ECGC (impurity-free chromatographs) after derivatization. The corresponding derivatives should be unambiguously separated (high resolution) on the μECGC, giving a high signal-to-noise ratio, optimally with an analysis time less than 5 minutes. Simple, straight chain aliphatic hydrocarbons and ethers generally fulfill the criteria for expense, purity and stability. Preferred detectable tags have the formula A or B:

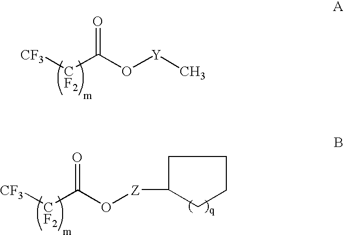

wherein

m is 4to 6;

Y is a saturated hydrocarbon of 12 to 32 carbons or a saturated hydrocarbon of 12 to 32 carbons in which one or more

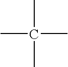

is replaced by —O—;

q is 1 to 3; and

Z is a saturated hydrocarbon of (9-q) to (29-q) carbons or a saturated hydrocarbon of (9-q) to (29-q) carbons in which one or more

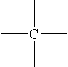

is replaced by —O—.

Potential structures of three classes of non-halogenated tags and their conversion to volatile derivatives are shown:

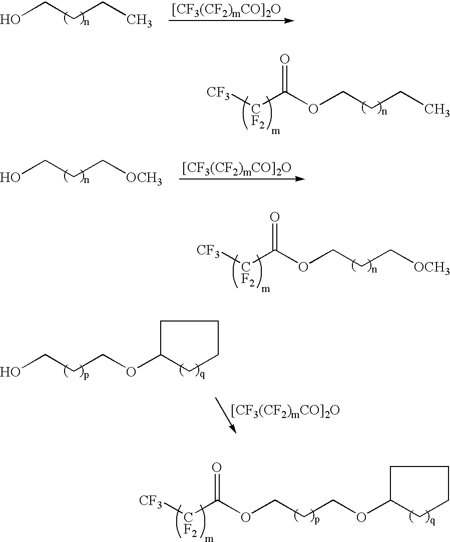

In these preferred compounds, m is zero to 10, optimally 4 to 6; n is 10 to 30, optimally 13 to 28; the sum of p and q is 10 to 30, optimally 13 to 28; and q will usually be one or two. The examples represent straight-chain alkyl in the form of —(CH₂)ₚ—, but branched and cyclic alkanes also work in the invention. Similarly, the use of ethers (oxaalkanols) provides an alternative embodiment of the invention, but in most cases the reagents are more expensive.

To attach the tags to the solid substrate through a linker, the tag alcohols are converted into a linker-tag complex by reaction of the activated alcohol with hydroxymethoxybenzoic acid followed by conversion to the corresponding diazoketones. The synthesis of the diazoketones described below. Polymer beads are then reacted with the diazoketones using the procedure described below.

The process of the invention allows reliable, routine decoding of tens of thousands of beads. A single bead in a master plate is incubated at room temperature for 2 h with 20 μL of aqueous 0.5 M CAN and 80 μL octane. The solution of the tags in octane is then treated with a huge excess of derivatizing reagent (commonly 500-fold excess or more) to rapidly and quantitatively derivatize the extracted tag alcohols. A large excess of derivatizing reagent is also employed to compensate for any surreptitious water that may be present in the octane leading to unwanted hydrolysis of derivatizing reagent. For this reason it is advantageous for the reagent to be inexpensive.

Volatizing detection reagents may be fluoroalkanoyl and haloaroyl anhydrides, halides, and activated esters. Condensing agents for reacting a cleaved tag having a terminal hydroxyl with a carboxylic acid are well known from the art of ester synthesis. Such agents include carbodiimides of various sorts, 2-ethoxy-1-ethoxycarbonyl-1,2-dihydroquinoline, O-(7-azabenzotriazol-1-yl)-1,1,3,3-tetramethyluronium hexafluorophosphate (HATU), and the like. It is also possible to pre-react the carboxylic acid of the detection reagent with an appropriate leaving group to form an activated ester, such as a triflate ester. Perfluoroalkanoyl anhydrides, particularly perfluoroheptanoic anhydride, are preferred.

Under certain circumstances it may be found that the use of an excess of derivatizing reagent overwhelms the detector, preventing accurate analysis. This may be overcome by decreasing the amount of reagent, although this may lead to unacceptably slow or incomplete derivatization. An alternative is the use of a scavenger resin to remove the excess derivatizing reagent from the octane solution prior to ECGC analysis. The scavenger can be any common resin that possesses a free hydroxyl group or amine group, enabling it to react with the excess derivatizing reagent.

Chemical reactions that are compatible with the strategy of the invention include condensation, amide bond formation, reduction, oxidation, elimination, substitution, alkylation, acylation, nitration, reductive amination, thiol addition, decarboxylation, dehalogenation, metal-halogen exchange and carbon-carbon coupling.

Optimally, the attachment of the tag to the solid substrate is accomplished by means of a linker interposed between the tag and the solid substrate. Usually it is advantageous to first attach the linker to the tag to form a linker/tag, and then attach the linker/tag to the solid substrate. Detachment can then be accomplished by oxidation, acid-catalyzed hydrolysis or photolytic decomposition of the linker. Suitable linkers are disclosed in U.S. Pat. No. 5,789,172. The 4-[4-(hydroxymethyl)-3-methoxyphenoxy]butyryl residue is a known linker, which is attached to a solid support having amino functionalities by forming an amide with the carboxyl of the butyric acid chain. The alkanol (or oxaalkanol) tags may be attached to the hydroxyl of the 4-hydroxymethyl group to form 2,4-dialkoxybenzyl ethers, which can be readily cleaved in acid media when the synthesis is complete. Preferred linkers are ortho- and para-nitrobenzyl ethers, which are cleaved photolytically, and ortho- and para-methoxyphenyl ethers, which are cleaved oxidatively.

Preferred tags are alkane and ether residues, preferably C₁₃ to C₃₃ alkanes and ethers, more preferably C₁₅ to C₃₀ alkanes and ethers as described above. Preferred linker/tag combinations are those of formula

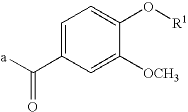

wherein

R¹ is C₁₃ to C₃₃ alkane or ether; and

a is the point of attachment to the solid phase support.

The linker/tags are synthesized by:

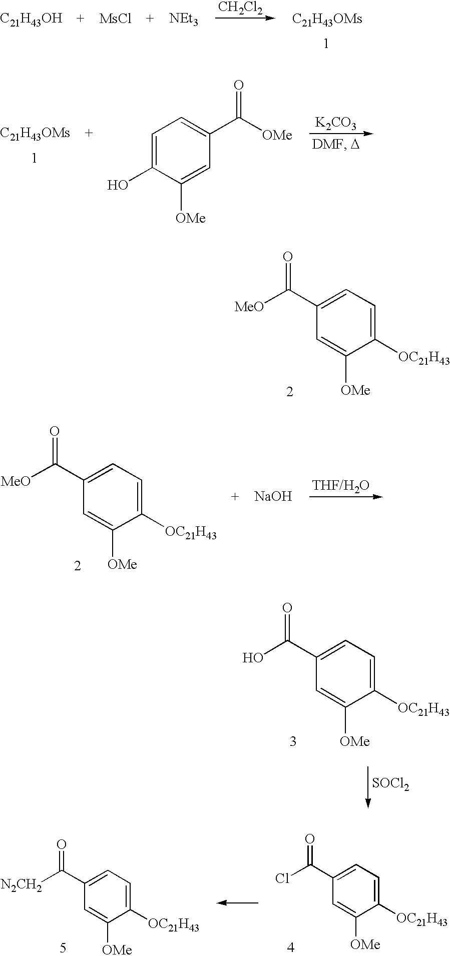

Synthesis of (1): To the alcohol, heneicosanol, (5 g, 16 mmol), suspended in 100 mL of CH₂Cl₂, was added methanesulfonyl chloride, (1.7 mL, 22 mmol) and triethylamine (3.5 mL). The solution was stirred at room temperature for 16 h. The reaction mixture was washed with water (2×) and the organic layer dried (Na₂SO₄), filtered and concentrated affording the mesylate (1) (6.4 g, 100% yield). This product was carried on to the next reaction without further purification.

Synthesis of (2): To a solution of (1) (6.4 g, 16 mmol) in 110 mL of dimethylformamide (DMF) was added methyl vanillate, (2.91 g, 16 mmol) and K₂CO₃ (16 g). The mixture was stirred at 45° C. for 40 h. After 40 h, the mixture was cooled to room temperature and 4N HCl was added to pH<4. The product was extracted with CHCl₃ (2×150 mL), washed with H₂O, dried (Na₂SO₄), filtered and concentrated to afford compound (2), (5.9 g, 77% yield) as a light yellow oil.

Synthesis of (3): To a solution of (2) (5.9 g, 12.9 mmol) in 100 mL of 30% H₂O/THF, was added NaOH pellets (9 g). The solution was heated at reflux for 40 h at which time a white precipitate had formed. The reaction mixture was allowed to cool to room temperature, and the supernatant was poured off. To the remaining solid was added 4N HCl with stirring until the pH remained<4. The solid was then filtered off and washed with IN HCl and H₂O and subsequently dried under reduced pressure at 60° C. for 8 h. The crude product (3) was isolated, (5.9 g, 100% yield).

Synthesis of (5): To a suspension of (3), (5.9 g, 12.9 mmol) in 100 mL toluene, was added thionyl chloride (4.8 mL, 65.8 mmol) and DMF (0.15 mL). The suspension was heated to give a clear solution. The solution was heated at reflux for 1.5 h. All volatiles were removed under vacuum. The resulting residue was dried under vacuum at 60° C. for 6 h. To a solution of the crude (4) in CHCl₃ (100 mL), at 0° C., was added triethylamine (4.1 mL). After 10 min, (trimethylsilyl)diazomethane (13 mL) was added. The solution was stirred at 0° C. for 1 h, after which it was allowed to warm to room temperature and stirred for 16 h. The reaction mixture was concentrated and the residue purified via flash chromatography (15:85 ethyl acetate:hexane) to give the diazo-linker/tag (5) (2.2 g, 35% yield).

Attachment of the linker/tag to the resin is accomplished as follows:

Before tagging the resin should be washed following the protocols outlined below:

For TENTAGEL™, ARGOGEL™, and polystyrene (200-250 μM) resins, wash 0.9 to 4 g of resin once with 60 mL methanol then five times with 60 mL of dichloromethane (DCM); for polystyrene resin (400-500 μM), wash 0.9 to 4 grams of resin five times with 60 mL DCM. The amount of diazo-linker/tag should be: for TENTAGEL™, ARGOGEL™, and polystyrene (200-250 μM) resins 7 to 15 mg of each tag diazoketone for each 100 mg of resin; for polystyrene resin (400-500 μM), 2 to 4 mg of each tag diazoketone for each 100 mg of resin. Dissolve the diazoketone in about 10 mL of DCM per vial, and 9 mg of rhodium trifluoroacetate dimer (ALDRICH) in 3 mL DCM per vial. Before addition of tag solution to vessels, small aliquots of each vial of tag solution should be saved for HPLC analysis. Suspend the washed resin in 45 mL DCM in a vial and add the diazoketone solution and the catalyst solution as follows:

i) add 33% of the catalyst solution to each vessel along with sufficient DCM to reach about 60 mL and shake for 30 minutes. Drain this solution from the vessel.

ii) add 33% of the catalyst solution to each vessel along with an appropriate amount of DCM so that 60 mL will be reached after all other additions. Shake for 10 minutes. Do not drain this solution from the vessel.

iii) add 30% of the tag solution to each vessel and shake for 10 minutes

iv) add another 30% of the tag solution to each vessel, shake for 10 minutes

v) add remaining 33% of the catalyst to each vessel, shake for 10 minutes.

vi) add the remaining 40% of the tag solution and the rinsings to each vessel, shake for 12 hours.

The reaction is monitored by HPLC to check for completion of tagging.

After encoding is completed, follow the procedure below:

For TENTAGEL™, ARGOGEL™, and polystyrene (200-250 μM) resins: The suspension is drained and washed with 60 mL of HPLC grade DCM (7×) and MeOH (6×) in an alternating manner. For polystyrene resin (400-500 μM): The suspension is drained and washed with 60 mL of DMF (5×) followed by DCM (8×).

The relative stability of an inert tag of the invention and of the polychlorophenoxyalkyl tags of U.S. Pat. No. 5,565,324 were compared in a prototype combinatorial synthesis employing the Suzuki Reaction:

Suzuki Coupling Reaction for Testing The Stability of the New Tags

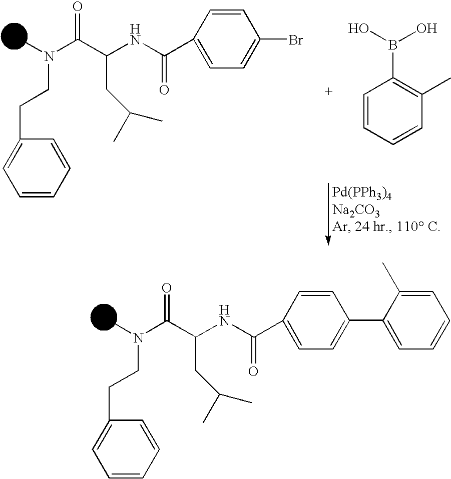

Suzuki reaction on solid support: In a 10 mL Merrifield shaking vessel was placed Polymer Labs 200-250 micron resin (5 mg) that had been previously encoded with the C₄ Cl₃, C₁₂ Cl₅, C₁₃ C₅ polychlorophenoxyalkyl tags of U.S. Pat. No. 5,565,324 and the C₁₇, and C₂₀ tags of the invention, followed by dimethoxyethane (DME) (5 mL), and the mixture was sparged with argon for 15 minutes. Phenyl boronic acid (129 mg, 1.54 mmol) was added followed by tetrabutyl ammonium hydroxide ( 0.78 g) and tetrakis triphenylphosphine palladium (0) (41 mg). The reaction mixture was shaken at 110° C. for 24 h and the resin then washed with DME (×2) followed by alternating cycles of dichloromethane and methanol (5 cycles). A number of beads were selected for detagging experiments to see if the reaction conditions had damaged either the tags of the invention or the prior art tags.

The cleavage and derivatization of a tag according to the invention are shown below:

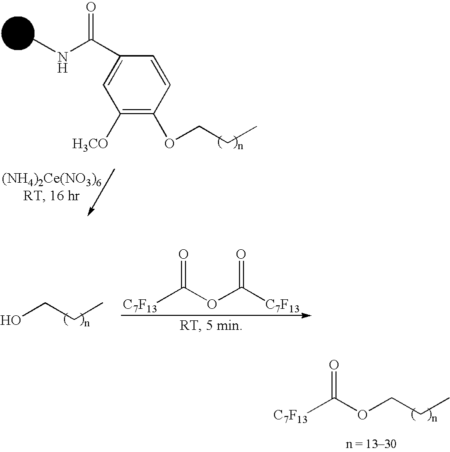

Extraction of tags of the invention and derivatization of the tags with perfluoroheptanoic anhydride as the volatizing detection reagent: To a glass insert containing a single tagged bead was added 2 μl of 0.3 M ammonium cerium (IV) nitrate (CAN) and 10 μl of octane. The insert was incubated at 30° C. for 16 hours. The octane solution was then transferred to another glass insert, followed by treatment with 2 μl of 0.2M perfluoroheptanoic anhydride octane solution and 1 μl of 1,1,1,3,3,3-hexamethyldisilazane (HMS). The reaction mixture then was dried under vacuum. The residue was re-dissolved in 8 μl of octane and 1 μl of HMS.

The experiment showed that the reaction conditions completely destroyed the prior art tags of the Cl₅ series. One microliter of solution was analyzed by GC, which showed the complete disappearance of the Cl₅ tags and the attenuation of the signal from the Cl₃ tag by 50% in comparison to the tags of the invention. The prior art tags of the Cl₃ series were thus damaged to the point of providing ambiguous information or no information. The tags of the invention were substantially unaffected. Substantially unaffected is a functional definition; it means that the tags were not sufficiently degraded to result in a loss of utility for identifying the solid substrate. Generally if a tag has suffered less than 5% loss or alteration in a series of reactions, it is “substantially” unaffected.

1. A method for identifying a solid support in combinatorial synthesis or determining the reaction history of a compound comprising attaching, detaching and identifying at least one alkane or oxaalkane tag, said identifying said tag being accomplished by reacting an alkanol or oxaalkanol obtained by detaching said tag from said solid support with a detection reagent chosen from fluoroalkanoyl acids and their alcohol-reactive equivalents.

2. A method according to claim 1 for identifying a solid support in combinatorial synthesis comprising:
(a) attaching at least one alkane or oxaalkane tag via an oxygen linkage to said solid support;
(b) carrying out at least one chemical reaction on said solid support having said tag attached;
(c) detaching said at least one tag from said solid support;
(d) reacting said detached tag with a detection reagent chosen from fluoroalkanoyl acids, anhydrides, halides, and activated esters, to provide an alkyl fluoroalkanoate or oxaalkyl fluoroalkanoate ester detectable tag; and
(e) detecting said tag, whereby said solid support is identified.

3. A method according to claim 1 of determining the reaction history of a compound, which comprises:
(a) providing a solid support on which a compound was synthesized by a reaction series comprising at least a first stage reagent and/or first stage reaction condition, and a second stage reagent and/or second stage reaction condition, wherein the first stage precedes the second stage in the reaction series, which solid support has attached thereto:
i. a first alkane or oxaalkane tag, which tag comprises a code that records the first stage reagent, the first stage reaction condition or both the reagent and the reaction condition; and
ii. a second alkane or oxaalkane tag, which tag comprises a code that records the second stage reagent, the second stage reaction condition or both the reagent and the reaction condition;
(b) detaching the tags from the solid support such that a mixture of alkanol or oxaalkanol tags is formed;
(c) reacting the mixture of tags with a detection reagent chosen from fluoroalkanoyl acids, anhydrides, halides, and activated esters to form a mixture of alkyl fluoroalkanoate or oxaalkyl fluoroalkanoate esters; and
(d) detecting each tag.

4. A method according to either of claims 2 or 3 wherein said detectable tag has the formula: 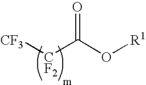
wherein
m is 4 to 6; and
R¹ is a saturated hydrocarbon of 13 to 33 carbons or a saturated hydrocarbon of 13 to 33 carbons in which one or more 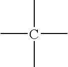
is replaced by —O—.

5. A method according to claim 4 wherein m is 6 and R¹ is a saturated hydrocarbon of 13 to 33 carbons.

6. A method according to either of claims 2 or 3 wherein said tag is covalently attached to the solid support through an intervening linker

7. A method according to claim 6 wherein said linker is first attached to said tag to form a linker/tag, and said linker/tag is then attached to said solid support.

8. A method according to claim 6 wherein said detaching is accomplished by oxidation, acid-catalyzed hydrolysis or photolytic decomposition of said linker.

9. A method according to claim 6 wherein said linker is chosen from the group consisting of ortho- and para-nitrobenzyl ethers and ortho- and para-methoxyphenyl ethers.

10. A method according to claim 9 wherein said linker and said tag together form a linker/tag residue of formula 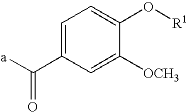
wherein
R¹ is C₁₃ to C₃₃ alkane or oxaalkane; and
a is the point of attachment to the solid phase support.

11. A method according to either of claims 2 or 3 wherein the detection reagent is a perfluoroalkanoic acid anhydride.

12. A method according to claim 1 wherein said identifying said tag is accomplished by electron capture gas chromatography.

13. A compound of formula 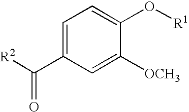
wherein
R¹ is C₁₃ to C₃₃ alkane or oxaalkane; and
R² is chosen from —CHN₂, —OH, halogen, —O-succinimide and —O-pentafluorophenyl.
